(19) United States
(12) Patent Application Publication
(10) Pub. No.: US 2010/0063565 A1
(43) Pub. Date: Mar. 11, 2010

(54) Skin Treatment Device

(75) Inventors: Frank Beerwerth, Kaltenholzhausen (DE); Brigitte Harttmann, Niedemhausen (DE); Dmitri Bogatirsky, Frankfurt/Main (DE)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: Braun GmbH, Kronberg (DE)

(21) Appl. No.: 12/515,389

(22) PCT Filed: Nov. 14, 2007
(86) PCT No.: PCT/EP07/09828

§ 371 Date: Aug. 24, 2009

(30) Foreign Application Priority Data
Nov. 18, 2006 (DE) 10 2006 054 468.4

(51) Int. Cl. A61N 5/06

(52) U.S. Cl. 607/88

(57) ABSTRACT

A skin treatment device for irradiating a skin area with electromagnetic radiation. The skin treatment device has an elastic at least partially hollow ring structure for leak-proof placement of the skin treatment device onto the skin. In another embodiment, the skin treatment device has a handle portion including a radiation source capable of emitting the electromagnetic radiation and a detachably mounted attachment having the elastic at least partially hollow ring structure for leak-proof placement of the skin treatment device onto the skin. As the device itself or the attachment has an at least partially hollow ring structure, it can adapt nicely even to strongly curved skin topologies.

### TECHNICAL FIELD

The present invention is concerned with skin treatment devices for skin treatment by irradiation with electromagnetic radiation. The invention is also concerned with an attachment and a handle portion that form a skin treatment device and it is concerned with a kit comprising the handle portion and at least an attachment.

### BACKGROUND

Skin treatment devices for treating skin with electromagnetic radiation, in particular with light, as such are known. Light-based devices are used by medical and aesthetical professionals for e.g. skin rejuvenation applications, for permanently removing hair, or for tattoo removal. For long-term hair removal via e.g. photothermolysis, hair root structures responsible for hair growth need to be damaged or destroyed. This can be accomplished by e.g. applying high-intensity light to the human skin that is absorbed by melanin in the papilla and the bulge of the hair follicles and thereby heating the hair follicle.

European patent specification EP 1 058 569 B1 describes an apparatus for removing hair by applying heat to the skin and the hair follicles. The housing of the apparatus preferably has a sealing gasket made from any suitable flexible material such as soft rubber for sealing the contact between the housing and a skin surface so that heated air cannot leak from the sealed cavity. It is further described that the apparatus is adapted to be used with a plurality of differently shaped extenders, which extenders may also include such a sealing gasket attached to the end of the extender distal to the apparatus for better sealing of the contact region with the skin. The purpose of the extenders with different apertures is to adapt the device to different regions of skin of different organs, such as the face, the limbs and the like.

It is a disadvantage of the described device that the described sealed contact with the skin in strongly curved regions or on skin with bony structures underneath, e.g. on the shin and the ankle region, can only be achieved with an extender having a small aperture. In contrast, in a device that treats skin with radiation, a fixed illumination window may be provided and an extender having an aperture smaller than the illumination window could not be chosen.

### SUMMARY

The skin treatment device as described comprises a radiation source capable of (controllably) emitting electromagnetic radiation when the device is switched on, in particular radiation in the UV, IR and/or visible spectrum, to a skin area to be treated. In addition, the skin treatment device comprises an elastic and at least partially hollow ring structure for leak-proof placement of the skin treatment device onto the skin area to be treated. The ring structure has a ring-like contact surface (depending on the specific realization of the ring structure, the contact surface can be a central line of a ring structure having a curved cross section or a central surface, if the ring structure has a flattened top to contact the skin) for contacting the skin. In dependence on skin topology and on the pressure exerted on the device to press the elastic ring structure onto the skin, the elastic ring structure will deform and become compressed. The skin area not contacted by the contact surface of the ring structure inside the elastic ring structure defines the maximum skin area that may be treated by direct radiation. As the elastic ring structure is at least partially hollow, it has a high compression capability when compared to a solid ring structure. Hence, good sealing contact with the skin can also be achieved in more problematic, strongly curved regions of the anatomy, e.g. on the shin, around the ankle or in the underarm region, while the elastic ring structure also allows continuous sliding over the skin, as it elastically and reversibly adapts to all kinds of skin curvatures. The ring structure could be a circular or oval ring structure or it could have a triangular, rectangular or any kind of polygonal ring shape or could be a combination of linear and curved pieces forming the closed ring structure. Circular, oval and pentagonal or higher polygonal ring shapes usually have a better gliding property as triangular or rectangular ring shapes, as the linear pieces of the latter ring shapes may bend over when glided over the skin surface.

In another embodiment, the skin treatment device has a handle portion (where the handle portion can comprise a base station and a hand piece) for applying electromagnetic radiation, in particular radiation in the UV, IR and/or visible spectrum, to a skin area to be treated. The handle portion comprises a radiation source arranged to (controllably) emit electromagnetic radiation when the skin treatment device is switched on (in embodiments with a base station, the radiation source can be located in the base station). In addition, the skin treatment device has a detachably mounted attachment that comprises an elastic and at least partially hollow ring structure for leak-proof placement of the skin treatment device (particularly, the radiation emitting head portion of the handle portion) onto the skin area to be treated. In addition to the advantages and benefits of an at least partially ring structure as described above, the attachment allows continuous sliding over the skin, as it adapts to all kinds of skin curvatures without the need to exchange the attachment as would be necessary in the prior art. As it also may be desirable to use the skin treatment device together with a lotion, the ring structure could easily become soiled after a certain number of usages. Additionally or alternatively, the elastic ring structure could become yellow and/or brittle under the influence of the high-intensity radiation. Further, in case of various users of the device, each user would want to have her/his own attachment for hygienic reasons. As the ring structure is arranged as a detachable attachment, it can easily be removed and replaced, irrespective of whether the replacement is for exchanging a soiled attachment by a fresh one or for attaching another user specific attachment. As described above, the ring structure could be a circular or oval ring structure or it could have a triangular, rectangular or any kind of polygonal ring shape or could be a combination of linear and curved pieces forming the closed ring structure. Circular, oval and pentagonal or higher polygonal ring shapes usually have a better gliding property than triangular or rectangular ring shapes, as the linear pieces of the latter ring shapes may bend over when glided over the skin surface.

Examples of partially hollow ring structures are rings having only a solid skin and a hollow interior cavity or a ring having a plurality of connected or unconnected, evenly or statistically distributed hollow cavities, e.g. a blistered interior. In one embodiment, the ring structure is made of an elastomer. In another embodiment, the ring structure is made of compressible foam. The foam could have a liquid-proof cover or could have a solidified outer surface. Such foam could be flexible elastomer foam such as polyurethane foam. Even though it might be sensible to have a tight and/or sealed skin of the elastic ring structure at least in the potential contact surface of the ring structure, specifically as the skin treatment device may be used with a lotion to enhance the gliding capability and/or to enhance the effect of the radiation used for the skin treatment, the elastic ring structure may have an irregularly formed skin surface, such as a foam having open blisters in the surface region. The ring structure may also have a specifically treated contact surface and /or outer skin, e.g. the skin could be coated with a material having a low friction coefficient when glided over the skin or the skin could be treated to be water repellent.

Leak-proof placement means that the ring structure ensures that the radiation cannot directly leak through the ring structure into the exterior of the device when the ring structure is in sealing contact with the skin. Typically, the ring structure would be designed to have dimensions that ensure that indirect light of high intensity, e.g. laser light that is scattered in the skin, cannot reach any person's eyes, where the dimensions are typically chosen so that national regulations such as the German DIN VDO 0837 are fulfilled.

In an embodiment of the skin treatment device (irrespective of whether the elastic ring structure is an integral part of the device or is arranged at an attachment), the skin treatment device has an illumination window that is arranged so as to be surrounded by the elastic ring structure. As the ring structure ensures a leak-proof placement of the skin treatment device onto a skin area even if the skin area is uneven or has bony structures underneath, it is thus avoided that radiation, e.g. high-intensity laser light, is leaking out from the device and reaches a user's or other person's eye or the like.

In a further embodiment of the skin treatment device with attachment it has a usage indicator that indicates a recommendation or a necessity to exchange the attachment, where the usage indicator could be triggered by e.g. integrated usage time, number of treatments or by an empty lotion cartridge. The usage indicator could be arranged either at the handle portion or at the attachment.

In a given embodiment of the skin treatment device, it has an applicator unit for applying a substance to the skin. Such substance could be a liquid, in particular a lotion that e.g. supports the envisaged skin treatment or cools the skin by an added coolant. A lotion could also improve the gliding properties of the attachment over the skin surface by an added lubricant. Nevertheless, the substance could also be a solid substance (e.g. soap or the like) that is abrasively applied to the skin. The applicator unit could also be a combination of a liquid dispenser and a solid substance applicator.

In another embodiment of the skin treatment device, a communication unit is arranged at least to provide information from the attachment to the handle portion, either wirelessly or in a wired manner. For providing a wired communication, the attachment and the handle portion could have contact pads that establish a wired connection for information exchange and/or energy transfer in the mounted state of the attachment. In a particular embodiment, the attachment could be provided with a specific code generator, which generates a code so that the skin treatment device will not work while the code is not received by the communication unit. This would improve secure handling of the skin treatment device, as it would ensure that without a proper attachment any radiation emission would not be initiated. Identifier information could also be provided as optically detectable markings on the attachment, such as a colour coding or a bar code. The handle portion could then be equipped with an optical detector to detect the information.

Another aspect includes a handle portion of a described skin treatment device, to which handle portion an attachment carrying the elastic ring structure can be detachably mounted via handle portion mounting means to form the skin treatment device.

In an embodiment, the handle portion has a handle portion part of a communication device for detecting information from the attachment and/or receiving or transmitting energy between the handle portion and the attachment. In an extended embodiment, the handle portion part of the communication device is coupled with a control circuit for controlling the radiation source. This allows to e.g. control the radiation in such a manner that the radiation can only be switched on in case of a properly mounted attachment.

Another aspect includes an attachment of a described skin treatment device, which attachment carrying the elastic and at least partially hollow ring structure can detachably be mounted to the handle portion of the skin treatment device via attachment mounting means to form the skin treatment device.

In one embodiment, the attachment has a radiation transparent window for coupling to the radiation source of the handle portion. This window in the attachment serves to protect the optical parts of the handle portion from soiling.

In various embodiments, the attachment has a contact surface of the ring structure that is (in its uncompressed condition) planar, convex or concave. The various contact surfaces serve to optimally adapt the ring structure to different skin topologies, e.g. to large areas as on the upper legs, to concave areas like in the underarm region or to convex regions like on the shin. A concave contact surface supports adaptation to convex skin topologies, such as in the shin region, and a convex contact surface supports adaptation to concave skin topologies, such as the underarm region.

In another embodiment, the attachment has (as an integral part) an applicator unit for applying a substance to the skin. Different attachments could hence be provided with different substances optimised for use in different skin regions of the user's body or for use with different skin types such as dry or oily skin. In an extended embodiment, the attachment has a substance reservoir. The reservoir could be equipped with a fill level detector that triggers the usage indicator to indicate a necessity to exchange the attachment if the fill level is zero.

In a further embodiment, the attachment has an attachment part of a communication unit at least for providing information to the handle portion and/or for receiving energy from the handle portion. In one embodiment, the attachment part of the communication unit is arranged to provide a (unique) identifier information to the handle portion part of the skin treatment device in a mounted state of the attachment. Hence, the skin treatment device can be arranged as only activatable in case an attachment is properly mounted.

In an even further embodiment, the attachment has a usage indicator for indicating a recommendation to exchange the attachment.

In another embodiment, the attachment has differentiator means that allow distinguishing various attachments that are provided for specific skin areas or skin types (depending on an applicator substance provided by the attachment) or that shall be assignable to a given user.

Another aspect relates to a kit comprising a handle portion as described and at least one attachment as described.

Various aspects will be explained in detail with reference to several exemplary embodiments and to figures.

### BRIEF DESCRIPTION OF THE DRAWINGS

FIG. 1 is a drawing of a skin treatment device,

FIG. 2 is an artist's impression of the hand piece of a skin treatment device with an attachment having an elastic ring structure shown in a detached state,

FIGS. 3a, 3b are depictions of a frontal view and a cross sectional cut of one embodiment of an attachment with an elastic ring structure,

FIGS. 4a, 4b are depictions of a frontal view and a cross sectional cut of another embodiment of an attachment with an elastic ring structure, and

FIG. 5 is a depiction of a communication unit of the skin treatment device and of its interaction with a control circuit for the radiation source.

### DETAILED DESCRIPTION

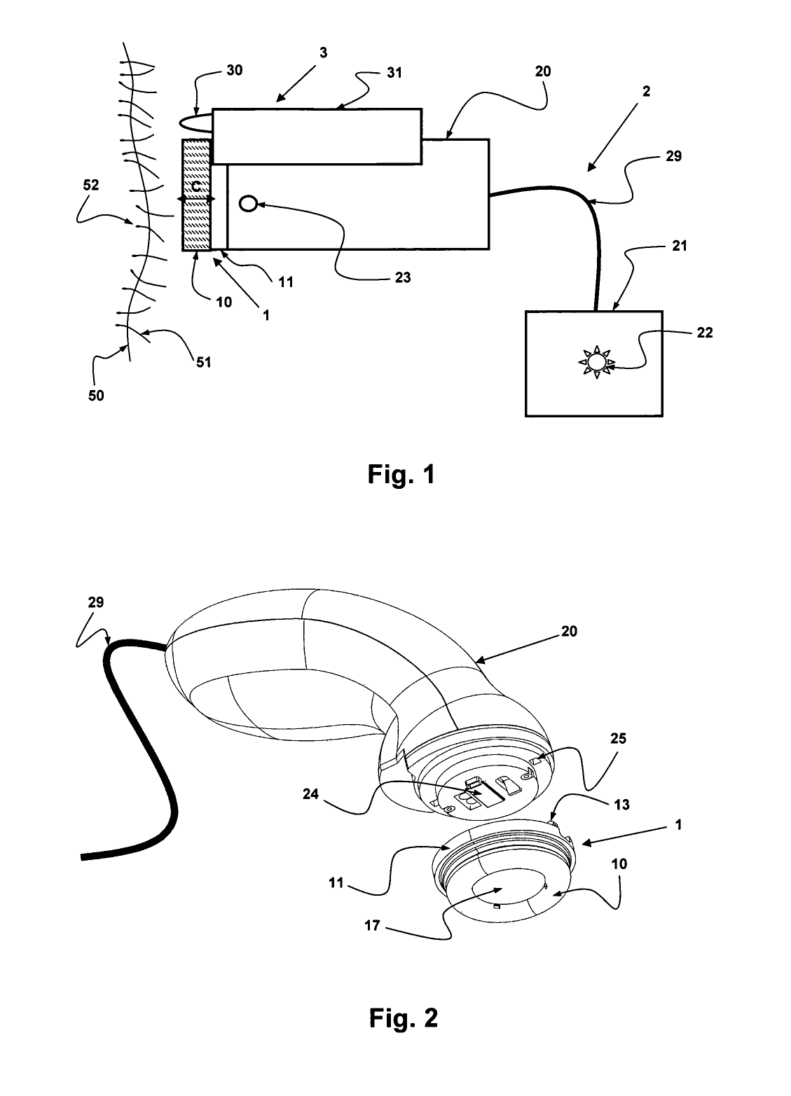

FIG. 1 shows a drawing of an exemplary skin treatment device comprising a handle portion 2 comprising a hand piece 20 and a base station 21, a front portion 1, and an applicator unit 3, where the front portion 1 could be an integral part of the skin treatment device or the front portion could be realized as a detachably mounted attachment. The applicator unit 3 could be an integral part of either the hand piece 20 of the handle portion 2 or of the front portion realized as an attachment 1 or the applicator unit 3 could be a detachably mounted unit that could be exchanged by a fresh applicator unit in case the used one is empty or could be replaced by a different applicator unit suitable for a different treatment (e.g. different application substances being applied for treating leg or underarm skin or for treating different skin types such as dry or oily skin). The handle portion 2 includes a base station 21 in which a radiation source 22 is located and a hand piece 20. The radiation (e.g. laser light) is transferred from the base station 21 to the hand piece 20 via a cable 29 that has a light-guide structure, e.g. a glass fibre bundle. The cable 29 could also comprise electrical connections for transfer of electrical energy and for data exchange. The front portion 1 includes an elastic and at least partially hollow ring structure 10. In case the front portion 1 is realized as a detachably mounted attachment, the attachment further comprises a base plate 11 for mounting the attachment to the hand piece 20 of the handle portion 2. The elastic ring structure 10 is compressible in direction C so that it can be placed on different skin topologies in a leak-proof manner ensuring that high intensity radiation cannot leak out when the radiation source 22 is switched on. In FIG. 1, also a skin surface 50 is indicated. Hairs 51 are growing on the skin. The hairs 51 have follicles 52 that will be at least damaged by applying the radiation. In contrast to the impression given by FIG. 1, it could be envisaged to shave the hairs 51 prior to the skin treatment to avoid any burning of hairs and the smell associated with it. In alternative embodiments, the skin treatment device is used for coagulating small vessels or small tissue volumes using focused radiation for improving the visual skin impression or the skin treatment device is used for any other known skin treatment. The skin treatment device, when realized with a detachably mounted attachment as front portion 1, further has a usage indicator 23 that indicates a recommendation and/or necessity for replacing the attachment. The usage indicator 23 in the shown embodiment is provided at the hand piece 20 but could alternatively be located at the base station 21 or at the attachment. In one embodiment, the usage indicator is a LED that switches from a green light to a red light to indicate the exchange recommendation and is triggered by a control circuit that integrates e.g. usage time or number of treatments.

Even though the elastic and at least partially hollow ring structure 10 could be an integral part (a not detachably mounted part) of the skin treatment device, the following description will specifically focus on embodiments of the skin treatment with a detachable attachment and the attachment itself. Naturally, all details pertaining to the elastic ring structure as a part of an attachment also apply to an elastic ring structure being an integral part of a skin treatment device without an attachment. In the following, reference numeral 1 will be assigned to the attachment as one specific realization of the front portion of a skin treatment device.

FIG. 2 shows an artist's impression of an exemplary hand piece 20 and an attachment 1, with the attachment 1 being depicted in a detached state. The hand piece 20 is connected via a cable 29 to a base station (as shown in FIG. 1) or to a power outlet, when the radiation source is housed in the hand piece 20 (in which case the cable 29 and the hand piece 20 would form the handle portion of the skin treatment device). The hand piece 20 has an exit window 24 for illuminating the skin with radiation. The hand piece 20 further has handle portion mounting means 25 arranged as recesses for receiving attachment-mounting means 13 that are arranged as clamping elements. The attachment 1 has a base plate 11 that carries an elastic ring structure 10 and an at least partially transparent window 17 that is provided to protect the illumination window 24 and sensor elements arranged around the illumination window 24 from any lotion used during the treatment, skin debris and skin fat. The elastic ring structure 10 has a circular contact surface to contact the skin and is designed in a two-part fashion. The part that will be pressed onto the skin surface essentially is a half torus that is connected via a second part formed as a bellow with the base plate 11. The elastic ring structure 10 in this embodiment is made from rubber or a latex material, but could generally be made from an elastomer.

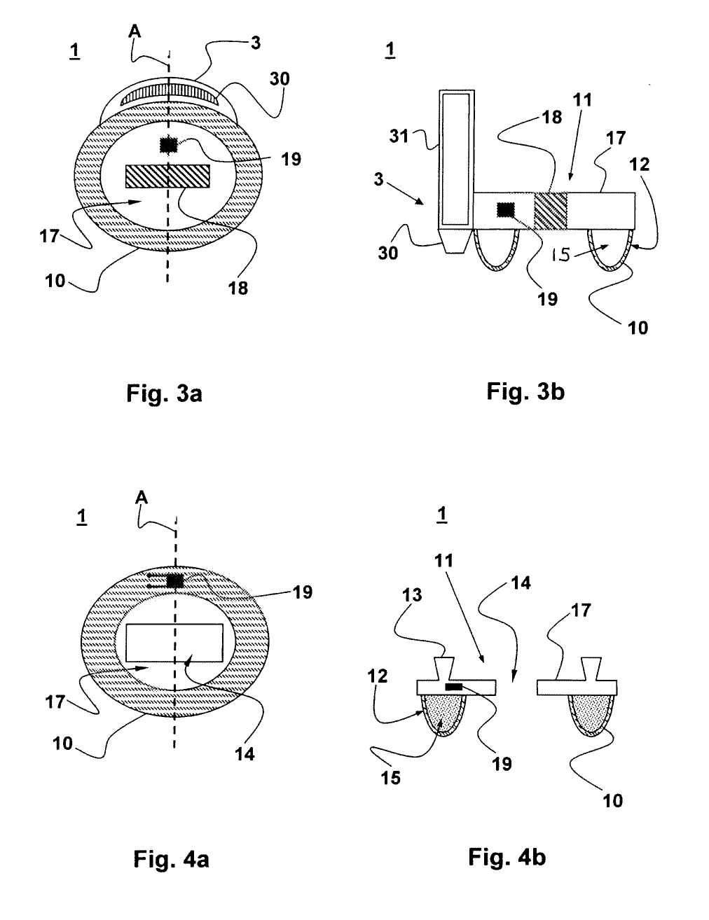

In FIG. 3a a frontal view onto one embodiment of an attachment is shown and FIG. 3b is a cross sectional cut through the attachment as shown in FIG. 3a along line A. The attachment 1 shown in FIG. 3a has an elastic ring structure 10 formed as an oval ring that surrounds a window 17, 18 having a radiation opaque portion 17 and a radiation transparent portion 18 that coincides in a mounted state with the illumination window 24 as shown in FIG. 2 of the handle portion so that radiation can be applied to the skin essentially without any absorption in the window. In an alternative embodiment, the window 17, 18 is completely transparent to avoid that backscattered light is absorbed and heats the window 17, 18. In another embodiment, the window 17, 18 is realized as a thin foil, e.g. made from a plastic material such as polycarbonate (PC). As an integral part of the attachment 1, an applicator unit 3 is provided that has a dispenser portion 30 for applying a substance to the skin. With reference to FIG. 3b, the applicator unit 3 has a substance reservoir 31, which in this embodiment is a liquid reservoir for storing an amount of lotion to be used during the skin treatment, and a substance dispenser 30, which in this embodiment is a spray for applying droplets of lotion onto the skin surface. In an alternative embodiment, the dispenser unit 30 is a roller for applying a lotion to the skin as is known e.g. from roll-on deodorants. In another embodiment, the applicator unit 3 has a block of a solid substance (e.g. a soap-like substance) that is pre-stressed against the skin and where the substance is abrasively applied to the skin. With continued reference to FIG. 3b, the attachment 1 has a base plate 11 to which the applicator unit 3 is connected. The base plate has a radiation transparent portion 18 and an opaque portion 17. The base plate 11 further carries the elastic ring structure 10, which is made of a skin part 12 surrounding a hollow cavity 15. The skin part is radiation opaque to assure that radiation cannot leak through the ring structure 10 when the attachment 1 is pressed against a skin area. In the present embodiment, the skin part 12 of the ring structure is made of rubber or latex material or any other suited elastomer. The elastic skin part 12 together with the hollow cavity 15 of the ring structure 10 assures very good local compressibility and the ring structure can fit leak-proof to even strongly curved parts of the user's anatomy, e.g. the shin, the ankle or the underarm. It is to be noted that the ring structure could have holes in the inside facing wall of the ring structure 10, e.g. for accommodating airflow between the hollow cavity inside the ring structure 10 and the cavity that is formed by the ring structure, the base plate 11 and the skin. Airflow from the hollow cavity inside the ring structure 10 to the exterior clearly being necessary for allowing high compressibility of the ring structure 10.

In the embodiment shown in FIG. 3a and FIG. 3b, the attachment 1 is equipped with an attachment part of a communication unit 19, which is realized as an RFID chip that can communicate with a handle portion part of a communication unit. Here, the attachment part of the communication unit 19 is moulded into the radiation opaque part 17 of the base plate 11. In order to prevent any heating of the attachment part of the communication unit 19, it could be placed elsewhere at the attachment, specifically in areas lying outside the directly or indirectly illuminated parts. In particular, the attachment part of the communication unit 19 could also be placed in a sidewall of the applicator unit 3.

In FIG. 4a, a frontal view of another embodiment of an attachment is shown and the corresponding cross sectional cut along line A is presented in FIG. 4b. The attachment 1 has a base plate of which the radiation opaque window 17 is visible in the frontal view (it needs to be noted that it is not necessary to have a radiation opaque window 17—in particular it could be recommendable to have a fully radiation transparent window 17 to avoid any heating of the window by absorbed indirect radiation). The window 17 has a cutout 14 that coincides with the illumination window 24 as shown in FIG. 2 in the mounted state or has somewhat larger dimensions. In the cross sectional cut shown in FIG. 4b, the base plate includes the window 17 with the cut-out 14 and attachment mounting means 13 formed as clamping elements that will be received by respective handle portion mounting means in the handle portion. In alternative embodiments, the attachment mounting means and the handle portion mounting means can take any form conceivable by a skilled person. The attachment mounting means could be formed as a screw thread that would then coincide with a respective negative screw thread at the handle portion. Alternatively, the attachment mounting means could be formed as a collar dimensioned to force-fit around a respective part of the handle portion. The attachment could also be arranged for crimping the attachment to the handle portion. The base plate 11 carries the elastic ring structure 10 that has a liquid-proof skin 12 and a cavity 15 filled with a compressible foam. As the elastic foam filling ensures that the ring structure 10 will reversibly take its original form after it was compressed, the skin 12 could be made thin. In an alternative embodiment, the foam solidifies in the skin region. In another embodiment, elastic foam without a skin part is used. As an elastic foam has a blistered structure (which means that a foam is an essentially hollow material), it features good local compressibility properties and hence will adapt to arbitrary skin topologies. In some embodiments, the foam is treated to be water repellent or has a coating having a low friction when glided over skin.

In the embodiment shown in FIGS. 4a and 4b, the attachment 1 again is equipped with an attachment part of a communication unit 19, where in the shown embodiment, the attachment part of the communication unit 19 has contacts that coincide with respective contacts of the handle portion part of the communication unit in the mounted state, so that a wired connection is established.

Typical dimensions for a ring structure are: outer diameter 2-20 cm, thickness of the ring structure in axial direction 0.1-2 cm, and height of the ring structure 0.2-5 cm. In one embodiment, the outer diameter is about 5 cm, the thickness of the ring structure in axial direction is 1 cm and the height of the ring structure is 1 cm.

In order to provide optimal leak-proof placement onto even the most strongly curved skin topologies, the contact surface of the ring structures could deviate from a planar contact surface. For the described ring structures, specifically with reference to FIG. 2, a central line or central area along the top of the ring structure would get into contact with a planar surface without any compression of the ring structure. In alternate embodiments, the contact surface could be a convex or concave surface. A pre-shaped ring structure having a convex contact surface would even better adapt to the skin topology in the armpit and a pre-shaped ring structure having a concave contact surface would even better adapt to a skin topology as present on the shin or the ankle.

Even though the ring structure was usually depicted as having (in a rest state or an uncompressed state, respectively) a half oval cross section, the cross section of the ring structure could of course take any other suitable form as, e.g., a rectangular cross section, in particular with rounded down edges, a circular cross section, a polygonal cross section etc. To enable a simple differentiation means between different attachments, an attachment could have a colour coding. A certain colour would then belong to a given user and/or would indicate a specific attachment or a specific application substance provided by the attachment. Alternatively, different attachments could be differentiated by an icon arranged on some eye-catching area. The differentiation could be made by simple geometrical forms or by icons identifying the preferred use of the attachment (e.g. by showing a stylised underarm, a stylised shin or stylised leg).

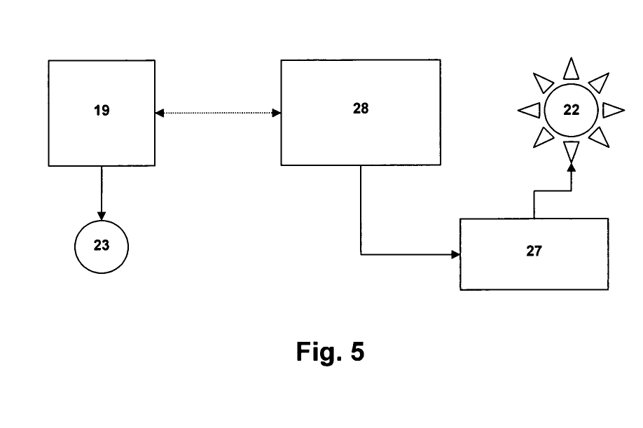

FIG. 5 shows a depiction of a communication unit 19, 28 of a skin treatment device and its interaction with a control circuit 27 for the radiation source. The attachment part of the communication unit 19 is adapted at least to provide information either wirelessly or by a wired connection to the handle portion part of the communication unit 28. The provision of the information could be implemented as a one-way communication (e.g. the attachment part of the communication unit only includes a transmitter for sending information) or as a two-way communication (receiver and transmitter included in each part of the communication unit). In a simple embodiment, the attachment part of the communication unit 19 provides (unique) identifier information to the handle portion part of the communication unit 28. The identifier information could be optically detectable information such as a colour coding or a bar code that is detected by an optical detector being part of the handle portion part of the communication unit 28. Only in case the identifier information is detected or received with the expected signal strength indicating that the attachment is correctly mounted to the handle portion, the handle portion part of the communication unit 28 will allow the control circuit 27 to switch on the radiation source. This assures that the skin treatment device can only be used with a mounted attachment and hence improves the secure usage of the skin treatment device. In another embodiment, the handle portion part of the communication unit 28 integrates the usage time of the skin treatment device and communicates the integrated value to the attachment part of the communication unit 19. In the shown embodiment, the usage indicator 23 is part of the attachment and the attachment part of the communication unit 19 is also adapted to receive information from the handle portion part of the communication unit 28. In case the integrated usage time exceeds a threshold value, the attachment part of the communication unit 19 will switch on the usage indicator to indicate a recommendation to exchange the attachment. The indication could e.g. be an optical or an acoustic signal.

It is to be noted that any use of “include” or “comprise” in the description or the claims shall mean “include, but not necessarily limited to”. Any use of “a” or “an” in the description or the claims in combination with a feature shall not limit this feature to be present only once but shall include also a multiple presence of the feature.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description are, in relevant part, incorporated herein by reference; the citation of any document is not to be construed as an admission that it is prior art. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications.

1-22. (canceled)

23. A skin treatment device for irradiating a skin area with electromagnetic radiation, the device comprising:
a hand piece;
a radiation source coupled to the hand piece and capable of emitting electromagnetic radiation; and
a skin-contacting component attachable to the hand piece and comprising a compressible, at least partially hollow ring structure arranged to form a leak-resistant contact with the skin.

24. The skin treatment device of claim 23, wherein the skin-contacting component is removably attachable to the hand piece.

25. The skin treatment device of claim 23, wherein the ring structure is elastic.

26. The skin treatment device of claim 23, wherein the skin-contacting component is attached to the hand piece and comprises an illumination window arranged in an opening of the ring structure.

27. The skin treatment device of claim 23, wherein the hand piece comprises a usage indicator operable to indicate an effective lifetime of the skin-contacting component.

28. The skin treatment device of claim 23, wherein the skin-contacting component comprises a usage indicator operable to indicate an effective lifetime of the skin-contacting component.

29. The skin treatment device of claim 23, further comprising an applicator unit operable to apply a substance to the skin.

30. The skin treatment device of claim 29, wherein the applicator unit defines a reservoir for containing a substance to be applied to the skin.

31. The skin treatment device of claim 29, wherein the skin-contacting component comprises the applicator unit.

32. The skin treatment device of claim 23, further comprising a communication unit capable of providing information from the skin-contacting component to another part of the skin treatment device.

33. The skin treatment device of claim 32, wherein the skin-contacting component comprises the communication unit.

34. The skin treatment device of claim 33, wherein the hand piece comprises a second communication unit capable of receiving information from the skin-contacting component.

35. The skin treatment device of claim 23, wherein the hand piece comprises a mount for removably attaching to the skin-contacting component, and the skin-contacting component comprises a mount for removably attaching to the hand piece.

36. The skin treatment device of claim 23, wherein the hand piece is coupled to a controller configured to control activation of the radiation source.

37. The skin treatment device of claim 23, wherein the skin-contacting component comprises an illumination window configured to allow radiation from the radiation source to irradiate the skin when the skin-contacting component is attached to the hand piece.

38. The skin treatment device of claim 23, wherein the ring structure comprises a planar skin-contacting surface.

39. The skin treatment device of claim 23, wherein the ring structure comprises a convex skin-contacting surface.

40. The skin treatment device of claim 23, wherein the ring structure comprises a concave skin-contacting surface.

41. The skin treatment device of claim 23, wherein the ring structure comprises an elastomer.

42. The skin treatment device of claim 23, wherein the ring structure comprises flexible foam.

43. The skin treatment device of claim 42, wherein the flexible foam is covered with a liquid-resistant elastic cover.

44. The skin treatment device of claim 42, wherein the flexible foam comprises a solidified outer layer for contacting the skin.

45. The skin treatment device of claim 23, wherein the skin-contacting component comprises an identifier selected from the group consisting of colors and icons.

46. A handle for a skin treatment device for irradiating an area of skin with electromagnetic radiation, the handle comprising:
a radiation source capable of emitting electromagnetic radiation; and
a mount configured to removably couple to a skin-contacting component capable of forming a leak-resistant contact with the skin.

47. An attachment for a skin treatment device for irradiating an area of skin with electromagnetic radiation, the attachment comprising:
a compressible, at least partially hollow ring structure arranged to form a leak-resistant contact with the skin; and
a mount for removably coupling to a handle capable of emitting electromagnetic radiation.

48. A kit comprising:
a handle comprising a radiation source capable of emitting electromagnetic radiation; and
an attachment configured to be removably coupled to the handle, the attachment comprising a compressible, at least partially hollow ring structure arranged to form a leak-resistant contact with the skin.
